(19) United States
(12) Patent Application Publication
(10) Pub. No.: US 2026/0056129 A1
(43) Pub. Date: Feb. 26, 2026

(54) METHOD FOR DETERMINING GLUCOSE CONCENTRATION IN THREE-DIMENSIONAL TUMOR SPHEROID IN VITRO

(71) Applicant: National Tsing Hua University, Hsinchu City (TW)

(72) Inventors: Fan-Gang TSENG, Hsinchu City (TW); Koyel DEY, Hsinchu City (TW)

(21) Appl. No.: 18/814,158

(22) Filed: Aug. 23, 2024

(51) Int. Cl. G01N 21/65; B82Y 15/00; C12N 5/09; G02B 21/34

(52) U.S. Cl. CPC G01N 21/658; C12N 5/0693; G02B 21/34; B82Y 15/00; C12N 2502/13; C12N 2513/00; C12N 2523/00; C12N 2533/30; C12N 2539/00

(57) ABSTRACT

A method for determining glucose concentration in a three-dimensional tumor spheroid in vitro includes: adding a surface-enhanced Raman spectroscopy (SERS)-based glucose nanosensor to a co-culture of cancer cells and stromal cells so as to form a first mixture; adding the first mixture into microwells of a polydimethylsiloxane (PDMS)-based microwell array chip; subjecting the first mixture in the PDMS-based microwell array chip to incubation at 37° C. so as to form the three-dimensional tumor spheroid with the SERS-based glucose nanosensor embedded therein; transferring the three-dimensional tumor spheroid onto a glass slide to allow the three-dimensional tumor spheroid to be mounted thereon; subjecting the three-dimensional tumor spheroid to confocal Raman spectroscopy so as to obtain Raman mapping images of the three-dimensional tumor spheroid; and extracting Raman intensities from the Raman mapping images and mapping the Raman intensities to a calibration curve so as to determine glucose concentration in the three-dimensional tumor spheroid in vitro.

### FIELD

The present disclosure relates to a method for determining glucose concentration in a tumor spheroid, and more particularly to a method for determining glucose concentration in a three-dimensional tumor spheroid in vitro.

### BACKGROUND

Cancer cells are capable of rapidly altering metabolic activities thereof, e.g., fermenting glucose to lactate even in the presence of oxygen, so as to meet insufficient oxygen supply and limited nutrients in the tumor microenvironment. Alteration in metabolic activities results in the increase in requirement of glucose, which prolonged the survival of cancer cells by increasing cell proliferation and protection against apoptosis. Thus, detection of glucose in the intratumor regions might provide crucial information on regulation of metabolism in the tumor microenvironment.

In recent years, multicellular tumor spheroid, which has a radial structure with a necrotic core surrounded by peripheral region containing proliferating cells and which is capable of forming gradients of oxygen, pH, glucose, and various other nutrients, has been developed as a three-dimensional (3D) tumor model for studying metabolism of nutrients and proliferation of tumor cells. For example, Bloch K. et al., in an article entitled “Metabolic alterations during the growth of tumor spheroids” published in Cell Biochem. Biophys., 2014, Vol. 68, p. 615-628, discloses the use of a 3D tumor model to characterize the utilization of glucose and to describe alterations to the activity and expression of key glycolytic enzymes during tissue growth. In addition, Goudar et al., in an article entitled “Impact of a desmoplastic tumor microenvironment for colon cancer drug sensitivity: a study with 3D chimeric tumor spheroids” published in ACS Appl. Mater. Interfaces, 2021, Vol. 13, p. 48478-48491, discloses that 3D co-culture of tumor spheroids formed on microwells of a 3D cell-culture array platform showed extracellular matrix interaction, intracellular communication, production of TGF-β1, type I collagen, and epithelium-to-mesenchymal transition (EMT) markers, etc., indicating the establishment of a robust desmoplastic tumor model. Therefore, a 3D tumor model mimicking tumor microenvironment in vivo can more accurately represent tumor physiology in vivo and cellular responses to drug therapy.

At present, methods for sensing glucose in 3D tumor model include the use of external electrochemical-based probes, photothermal optical coherence tomography (PTOCT), quantum dots, etc. In the case of PTOCT, surface-enhanced Raman spectroscopy (SERS)-based nanosensors were targeted to specific regions of tumor spheroids along with the application of an external laser source so as to generate photothermal signal. However, real-time monitoring of glucose in tumor spheroids using these techniques are difficult due to limitations such as cytotoxicity, interference with cellular autofluorescence, and tissue damage caused by application of photothermal or electromagnetic field.

It should be noted that SERS has been applied for analyzing glucose, pH and redox potential of biological samples due to the relatively high sensitivity of such technique which is attributed to the fingerprint of the molecules of interest. However, direct detection of biochemical compound by SERS is still challenging due to poor adsorption of such compound on the surfaces of metal substrates or nanostructures. Previous studies have reported the use of nanostructures, such as silver (Ag) or gold (Au) nanopillars-based substrates, as nanosensors which are capable of enhancing Raman signals by a factor ranging from 106 to 109. For example, Lyandres O. et al., in an article entitled “Real-time glucose sensing by surface-enhanced Raman spectroscopy in bovine plasma facilitated by a mixed decanethiol/mercaptohexanol partition layer” published in Anal. Chem., 2005, Vol. 77, p. 6134-6139, discloses development of a SERS-based glucose sensor prepared from Ag-coated polystyrene nanospheres that are mixed with decanethiol and mercaptohexanol. However, the linker molecules with a relatively large size have tendency to keep glucose molecules away from the nearfield enhancement of the surface of SERS substrate, resulting in difficulty to obtain consistent and precise measurement of glucose concentration. In addition, 4-Mercaptophenylboronic acid (4-MPBA) has been reported to display distinguishable Raman peaks upon binding to plasmonic nanoparticles or metal surface because mercapto group has high affinity to metals (e.g., Au and Ag), and the ability to selectively recognize monosaccharides such as glucose. Despite the numerous studies conducted to measure ambient glucose concentration ex vivo, continuous in vitro measurement of physiological parameters in 3D culture model still remains a challenge.

Therefore, there is an urgent need to develop a new strategy which allows continuous and rapid analysis of glucose distribution in 3D tumor models in vitro.

### SUMMARY

Therefore, an object of the present disclosure is to provide a method for determining glucose concentration in a three-dimensional tumor spheroid in vitro which can alleviate at least one of the drawbacks of the prior art.

According to the present disclosure, the method includes the steps of:
(a) adding a surface-enhanced Raman spectroscopy (SERS)-based glucose nanosensor including 4-mercaptophenylboronic acid (4-MPBA)-functionalized silver nanoparticles to a co-culture of cancer cells and stromal cells, so as to form a first mixture;
(b) adding the first mixture into microwells of a polydimethylsiloxane (PDMS)-based microwell array chip;
(c) subjecting the first mixture in the PDMS-based microwell array chip to incubation at 37° C. so as to form the three-dimensional tumor spheroid with the SERS-based glucose nanosensor embedded therein;
(d) transferring the three-dimensional tumor spheroid onto a glass slide to allow the three-dimensional tumor spheroid to be mounted thereon;
(e) subjecting the three-dimensional tumor spheroid to confocal Raman spectroscopy so as to obtain Raman mapping images of the three-dimensional tumor spheroid; and
(f) extracting Raman intensities from the Raman mapping images and mapping the Raman intensities to a calibration curve so as to determine glucose concentration in the three-dimensional tumor spheroid in vitro.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Other features and advantages of the present disclosure will become apparent in the following detailed description of the embodiment(s) with reference to the accompanying drawings. It is noted that various features may not be drawn to scale.

FIG. 1 is a flow diagram illustrating the steps in a method for determining glucose concentration in a three-dimensional tumor spheroid in vitro.

FIG. 2A shows a transmission electron microscopy image of the surface morphology of the 4-mercaptophenylboronic acid (4-MPBA)-functionalized silver (Ag) nanoparticles (NP).

FIG. 2B shows the ultraviolet-visible (UV-Vis) spectroscopy spectra of the Ag NP and the 4-MPBA-functionalized Ag NP, respectively.

FIG. 2C shows distributions of zeta potential of the Ag NP and the 4-MPBA-functionalized Ag NP.

FIG. 2D is a Raman spectra showing the Raman intensities of the Ag NP and the 4-MPBA-functionalized Ag NP under a wavenumber ranging from 600 cm⁻¹ to 1600 cm⁻¹.

FIG. 3A and FIG. 3B are graphs respectively showing the relative cell viability (%) of HCT-8 cells and that of NIH-3T3 cells when tested against 4-MPBA-functionalized Ag NP at a concentration ranging from 0.019 mg/L to 10.0 mg/mL.

FIG. 4A shows a fluorescence microscopy image of the fluorescein isothiocyanate (FITC)-conjugated Ag NP with a scale bar of 10 μm.

FIG. 4B shows the bright-field and dark-field confocal fluorescence microscopy images of the HCT-8 cells and those of the NIH-3T3 cells after incubation with the FITC-conjugated Ag NP, in which (i) and (ii) represent 4 hours of incubation, (iii) and (iv) represent 8 hours of incubation, (v) and (vi) represent 12 hours of incubation, and (vii) and (viii) represent 24 hours of incubation.

FIG. 5A is a diagram showing the chemical structure of the 4-MPBA-functionalized Ag NP, in which the 4-MPBA binds to the Ag NP via thiol bonding.

FIG. 5B is diagram showing the chemical structure of the 4-MPBA-functionalized Ag NP with the hydroxyl (—OH) groups of the 4-MPBA binding to a glucose molecule through a dehydration reaction.

FIG. 6A is a Raman spectra showing the Raman intensities for the various glucose solutions having concentrations ranging from 0.1 mM to 50 mM (only 6 mM, 7 mM, 8 mM, 9 mM, 10 mM, 25 mM and 50 mM were indicated) at a wavenumber ranging from 800 cm⁻¹ to 1400 cm⁻¹ after being tested against the 4-MPBA-functionalized Ag NP.

FIG. 6B is an enlarged view taken from the boxed region indicated by the dashed line in FIG. 6A which shows the Raman intensities for the aforesaid various glucose solutions having concentrations ranging from 0.1 mM to 5.0 mM at a wavenumber ranging from 800 cm⁻¹ to 1400 cm⁻¹ after being tested against the 4-MPBA-functionalized Ag NP.

FIG. 6C is a graph showing the Raman intensities at the wavenumber of 1078 cm⁻¹ plotted against a glucose concentration ranging from 0.1 mM to 50 mM.

FIG. 6D is graph showing the Raman intensities at the wavenumber of 1078 cm⁻¹ plotted against a glucose concentration ranging from 0.1 mM to 10.0 mM with a linear regression, R² of 0.96.

FIG. 7A is a Raman spectrum showing the Raman intensity at a wavenumber of 1078 cm⁻¹ for the PBS solution and the glucose solution after such solutions are sequentially tested 3 times against the 4-MPBA-functionalized Ag NP.

FIG. 7B is a graph showing the Raman intensity at the wavenumber of 1078 cm⁻¹ for the 4-MPBA-functionalized Ag NP at a temperature ranging from 20° C. to 50° C.

FIG. 7C is a graph showing the Raman intensity at the wavenumber of 1078 cm⁻¹ for the glucose solutions respectively containing 1 mM of glucose, 1 mM of glucose and 2 mM NaCl, 1 mM of glucose and 2 mM of KCl, 1 mM of glucose and 2 mM of CaCl₂), 1 mM of glucose and 2 mM of MgCl₂, and 1 mM of glucose and 2 mM of Ca(HCO₃)₂, after being tested against the 4-MPBA-functionalized Ag NP on the APTES-functionalized glass slides.

FIG. 8 is an scanning electron microscopy (SEM) image illustrating a top schematic view of several microwells of the PDMS-based microwell array chip with a scale bar of 100 μm.

FIG. 9A is a graph showing the cell number per twenty 3D tumor spheroids determined on day 0 to day 5.

FIG. 9B is a graph showing the average size of the 3D tumor spheroid (per twenty tumor spheroids) determined on day 1 to day 5.

FIG. 10A is an SEM image showing the 3D tumor spheroids formed in the microwells of the PDMS-based microwell array chip after incubating the HCT-8 cells, NIH-3T3 cells and 4-MPBA-functionalized Ag NP therein for 48 hours.

FIG. 10B is a fluorescence microscopy image of the 5-day old 3D tumor spheroid at maximum intensity projection with a scale bar of 50 μm.

FIG. 10C is a fluorescence microscopy image showing a cross-sectional view of the 5-day old 3D tumor spheroid which was taken from the boxed region indicated by the dashed lines in FIG. 10B.

FIG. 11 is an energy-dispersive X-ray spectroscopy (EDS) spectrum showing the elements present in the 3D tumor spheroids.

FIG. 12A shows confocal fluorescence microscopy images of a 3D tumor spheroid formed after incubation of the HCT-8 cells, NIH-3T3 cells and FITC-conjugated Ag NP for 5 days, in which (i) to (iv) respectively are the top portion, the first middle portion, the second middle portion and the bottom portion of the 3D tumor spheroid.

FIG. 12B shows confocal fluorescence microscopy images of the HCT-8 cells and the NIH-3T3 cells after disruption of the 3D structure of the 5-day old tumor spheroids, in which (i) is a bright-field image of the HCT-8 cells and NIH-3T3 cells, (ii) to (iv) are dark-field images of the HCT-8 cells and NIH-3T3 cells respectively stained with CellTracker™ Red CMPTX, DAPI and FITC dyes, and (v) is an image obtained by merging the images shown in (ii) to (iv).

FIG. 13A and FIG. 13B are graphs showing the Raman intensities extracted and determined from the Raman mapping images that were generated at the wavenumber bandwidth of 1078 cm⁻¹ for the core, quiescent and peripheral sections of the 3-day old 3D tumor spheroids and the 5-day 3D tumor spheroids, respectively.

FIG. 13C and FIG. 13D are graphs showing the Raman intensities extracted and determined from the Raman mapping images that were generated at the wavenumber bandwidth of 1078 cm⁻¹ for the core, quiescent and peripheral sections of the 3-day old 3D fibroblast spheroids and the 5-day old 3D fibroblast spheroids, respectively.

FIG. 14A is a graph showing the glucose concentrations determined in the core, quiescent and peripheral sections of the 3-day old 3D tumor spheroids and those of the 5-day old 3D tumor spheroids, in which the symbol “***” represents p<0.0001 with respect to comparison of the glucose concentration in the culture media for the 5 day-old 3D tumor spheroids with the glucose concentration in the culture media for the 3 day-old 3D tumor spheroids, and the Raman mapping image of the 5-day old 3D tumor spheroids (scale bar: 2 μm) indicating the distribution of Raman intensity at the wavenumber of 1078 cm⁻¹ was shown in the insert of this figure.

FIG. 14B is a graph showing the glucose concentrations determined in the core, quiescent and peripheral sections of the 3-day old 3D fibroblast spheroids and those of the 5-day old 3D fibroblast spheroids, in which the symbol “***” represents p<0.0001 with respect to comparison of the glucose concentration in the culture media for the 5 day-old 3D fibroblast spheroids with the glucose concentration in the culture media for the 3 day-old 3D fibroblast spheroids.

FIG. 15 is a Raman spectra showing the Raman intensities for the intracellular contents of the 3-day old 3D tumor spheroids and the 5-day old 3D tumor spheroids, respectively, under a wavenumber ranging from 800 cm⁻¹ to 1200 cm⁻¹.

### DETAILED DESCRIPTION

Before the present disclosure is described in greater detail, it should be noted that if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

In order to address the current limitations of 3D tumor model in determining glucose distribution in vitro, the applicants endeavored to develop improved methods and found that 4-MPBA-functionalized silver (Ag) nanoparticles (NP) can be applied as a SERS-based glucose nanosensor to be embedded in 3D tumor spheroids formed in a microwell array chip for continuously and rapidly detecting glucose in such 3D tumor spheroids in vitro, so as to allow measurement of glucose concentration in such tumor spheroids.

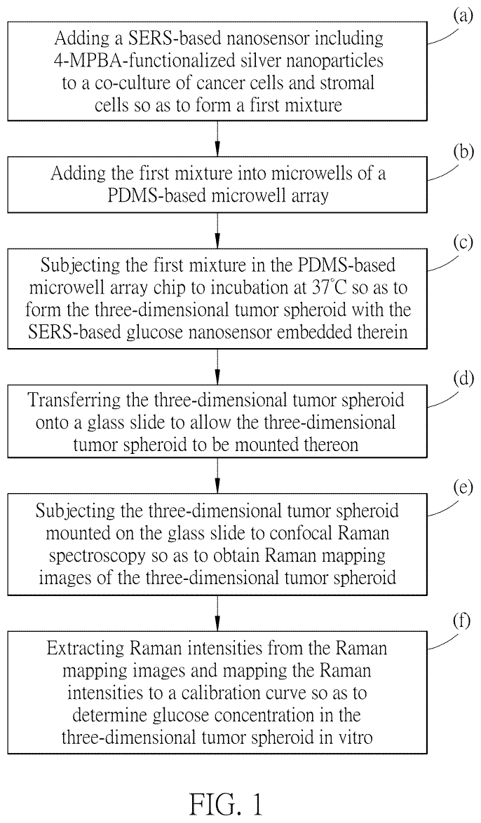

Referring to FIG. 1, a method for determining glucose concentration in a three-dimensional tumor spheroid in vitro according to the present disclosure includes the steps of: (a) adding a surface-enhanced Raman spectroscopy (SERS)-based nanosensor including 4-mercaptophenylboronic acid (4-MPBA)-functionalized silver nanoparticles to a co-culture of cancer cells and stromal cells so as to form a first mixture; (b) adding the first mixture into microwells of a polydimethylsiloxane (PDMS)-based microwell array chip; (c) subjecting the first mixture in the PDMS-based microwell array chip to incubation at 37° C. so as to form the three-dimensional tumor spheroid with the SERS-based glucose nanosensor embedded therein; (d) transferring the three-dimensional tumor spheroid onto a glass slide to allow the three-dimensional tumor spheroid to be mounted thereon; (e) subjecting the three-dimensional tumor spheroid mounted on the glass slide to confocal Raman spectroscopy so as to obtain Raman mapping images of the three-dimensional tumor spheroid; and (f) extracting Raman intensities from the Raman mapping images and mapping the Raman intensities to a calibration curve so as to determine glucose concentration in the three-dimensional tumor spheroid in vitro.

As used herein, the term “tumor spheroid” or “tumor cell spheroid” refers to an aggregation of tens-of-thousands of tumor cells constituting a small mass or lump of tumor cells with a spherical shape.

As used herein, the term “surface-enhanced Raman spectroscopy (SERS)” refers to a surface-sensitive sensing technique that enhances inelastic scattering of photons (with an enhancement factor up to 108 or even larger) by molecules adsorbed on rough or corrugated metal surfaces such as silver or gold nanoparticles.

According to the present disclosure, in step (a), examples of the cancer cells include, but are not limited to, colon cancer cells, breast cancer cells, lung cancer cells, ovarian cancer cells, pancreatic cancer cells, prostate cancer cells, skin cancer cells, bone marrow cancer cells, brain cancer cells, gastrointestinal cancer cells, renal cancer cells, liver cancer cells, testicular cancer cells, bladder cancer cells, cervical cancer cells, esophageal cancer cells, and endometrial cancer cells, and examples of the stromal cells include, but are not limited to, endothelial cells, macrophages, fibroblasts, mesenchymal stem cells, osteoblasts, chondrocytes, and myocytes.

In an exemplary embodiment, the colon cancer cells are human ileocecal adenocarcinoma HCT-8 cells, and the fibroblasts are mouse embryonic fibroblast NIH-3T3 cells.

In an exemplary embodiment, a ratio of a number of the human ileocecal adenocarcinoma HCT-8 cells to a number of the mouse embryonic fibroblast NIH-3T3 cells is 1:1.

In certain embodiments, in step (a), a concentration of the SERS-based glucose nanosensor added to the co-culture ranges from 0.019 mg/ml to 10.0 mg/mL.

In an exemplary embodiment, in step (a), the concentration of the SERS-based glucose nanosensor added to the co-culture is 10.0 mg/mL.

In certain embodiments, in step (c), the first mixture in the PDMS-based microwell array chip is incubated at 37° C. for a time period ranging from 48 hours to 120 hours.

In an exemplary embodiment, in step (c), the first mixture in the PDMS-based microwell array chip is incubated at 37° C. for 72 hours.

In another exemplary embodiment, in step (c), the first mixture in the PDMS-based microwell array chip is incubated at 37° C. for 120 hours.

According to the present disclosure, the 4-MPBA-functionalized silver nanoparticles are prepared by the steps of: (i) heating a silver nitrate solution at 90° C. under stirring to obtain a boiled silver nitrate solution; (ii) adding dropwise a sodium citrate solution to the boiled silver nitrate solution under stirring so as to form a yellow-colored solution; (iii) cooling the yellow-colored solution to room temperature until a colloidal solution containing silver nanoparticles was formed; (iv) adding a 4-MPBA solution in ethanol, which is prepared by mixing 4-MPBA and ethanol, to the colloidal solution containing the silver nanoparticles under stirring so as to form a second mixture; and (v) incubating the second mixture in the dark at 4° C. to allow the 4-MPBA to be conjugated to the silver nanoparticles, so as to obtain the 4-MPBA-functionalized silver nanoparticles.

In certain embodiments, in step (ii), the sodium citrate solution is added dropwise to the boiled silver nitrate solution for a time period ranging from 1 minute to 15 minutes.

In an exemplary embodiment, in step (ii), the sodium citrate solution is added dropwise to the boiled silver nitrate solution for 15 minutes.

In certain embodiments, in step (iv), a weight ratio of the silver nanoparticles to the 4-MPBA ranges from 533:1 to 650:1.

In an exemplary embodiment, in step (iv), a weight ratio of the silver nanoparticles to the 4-MPBA is 650:1.

In certain embodiments, in step (v), the second mixture is incubated in the dark for a time period ranging from 8 hours to 12 hours.

In an exemplary embodiment, in step (v), the second mixture is incubated in the dark for 12 hours.

In certain embodiments, the 4-MPBA-functionalized silver nanoparticles have a particle size ranging from 40 nm to 60 nm.

In certain embodiments, the 4-MPBA-functionalized silver nanoparticles have an average particle size of 50 nm.

The present disclosure will be described by way of the following examples. However, it should be understood that the following examples are intended solely for the purpose of illustration and should not be construed as limiting the present disclosure in practice.

### EXAMPLES

### General Experimental Materials

### 1. Chemicals

Silver nitrate (AgNO₃; purity: >99%) was purchased from Sigma Aldrich, USA. Sodium citrate (Na₃C₆H₅O₇; purity: >99%) was purchased from Choneye Co., Ltd., Taiwan. 4-Mercaptophenylboronic acid (4-MPBA; purity: 90%) was purchased from Combi-Blocks, USA. Ethanol (purity: 99.5%) was purchased from Echo Chemical, Taiwan. An epoxy-based photoresist, SU-8 2150, was purchased from Microchem Corp., USA. A silane coupling agent, 1H,1H,2H,2H-perfluorooctyltrichlorosilane (purity: 97%), was purchased from Alfa Aesar, USA. Polydimethylsiloxane (PDMS) prepolymer, (Sylgaurd® 184 Silicon Elastomer) was purchased from Dow Corning Corp., USA. Pluronic® F-127 was purchased from Merck, China. (3-Aminopropyl)triethoxysilane was purchased from Sigma Aldrich, USA. D-(+)-Glucose (C₆H₁₂O₆; purity: ≥99.5%; CAS No.: 50-99-7) was purchased from Sigma Aldrich, USA. Phosphate-buffered saline (PBS) was purchased from Gibco, USA. Methanol (purity: 99%) was purchased from Echo Chemical, Taiwan. Fluorescein isothiocyanate (FITC; purity: >90%) was purchased from Sigma Aldrich, USA. Trypsin-disodium ethylenediaminetetraacetic acid (EDTA) was purchased from Gibco, USA. Triton-X 100 was purchased from Sigma Aldrich, USA. 4′,6-diamino-2-phenylindole (DAPI) dye was purchased from Thermo Fisher Scientific, USA. CellTracker™ Red CMTPX dye and CellTracker™ Green CMFDA dye were purchased from Thermo Fisher Scientific, USA.

### 2. Source and Cultivation of Cells

Human ileocecal adenocarcinoma HCT-8 cells and mouse embryonic fibroblast NIH-3T3 cells were purchased from Bioresource Collection and Research Center (BCRC) of the Food Industry Research and Development Institute (FIRDI), Hsinchu, Taiwan under BRCR accession numbers 60303 and 60008, respectively. The HTC-8 cells were incubated in cell culture dish containing RPMI 1640 medium (Manufacturer: Dow Corning Corp., USA) supplemented with fetal bovine serum (FBS), while the NIH-3T3 cells were incubated in cell culture dish containing Dulbecco's modified Eagle's medium (DMEM) (Manufacturer: Dow Corning Corp., USA) supplemented with FBS, followed by cultivation in an incubator with culture conditions set at 37° C. and 5% CO₂. Medium change was performed every two days. Cell passage was performed when the cultured cells reached 70% of confluence.

### General Experimental Procedures:

### 1. Statistical Analysis

All the experiments described below were performed in triplicates. Statistical analysis was conducted using GraphPad Prism 6 software (Developer: GraphPad Sofware, Inc., San Diego, CA). The experimental data of all the test groups are expressed as mean±standard deviation (SD), and were analyzed using one-way analysis of variance (ANOVA) with Bonferroni correction, so as to assess the differences between the groups. Statistical significance is indicated by p<0.05.

### Example 1. Preparation and Evaluation of 4-Mercaptophenylboronic Acid (4-MPBA)-Functionalized Silver Nanoparticles (Ag NP)

In this example, 4-MPBA-functionalized Ag NP were prepared and then subjected to the following evaluations, followed by utilization of the same as a surface-enhanced Raman scattering (SERS) substrate for detecting glucose in solutions.

### A. Preparation of 4-Mercaptophenylboronic Acid (4-MPBA)-Functionalized Ag NP

The 4-MPBA-functionalized Ag NP were prepared with reference to an article by Pham X.-H. et al., in an article entitled “Glucose detection using 4-mercaptophenyl boronic acid-incorporated silver nanoparticles-embedded silica-coated graphene oxide as a SERS substrate” published in BioChip J., 2017, Vol. 11, p. 46-56. First, an AgNO₃ solution (1.0 mM) was heated at 90° C. with stirring at 400 rpm to obtain a boiled AgNO₃ solution. Immediately thereafter, a sodium citrate solution (1% w/v) was added dropwise to the boiled AgNO₃ solution under stirring at 500 rpm for 15 minutes or until a yellow-colored solution is obtained, indicating the formation of Ag NP. Next, the yellow-colored solution was cooled to room temperature which might range from 16.5° C. to 30.5° C. until a colloidal solution containing Ag NP was formed. Then, 5 μl of a 4-MPBA solution (2 mg/ml) in ethanol, which was prepared by mixing 4-MPBA and ethanol (purity: 99.5%), was added to 200 μL of the colloidal solution (32.5 mg/mL) containing Ag NP under stirring so as to form a mixture, followed by incubating the mixture in the dark at 4° C. for 12 hours to allow the 4-MPBA, through the thiol group thereof, to be conjugated to the Ag NP, thereby obtaining the 4-MPBA-functionalized Ag NP. After that, the 4-MPBA-functionalized Ag NP were washed with deionized water to remove non-conjugated 4-MPBA residues, and then resuspended in fresh deionized water.

### B. Evaluation of 4-Mercaptophenylboronic Acid (4-MPBA)-Functionalized Ag NP

The Ag NP (before conjugation to the 4-MPBA) and the 4-MPBA-functionalized Ag NP obtained in section A above were subjected to property evaluation described hereinafter.

### 1. Determination of Surface Morphology

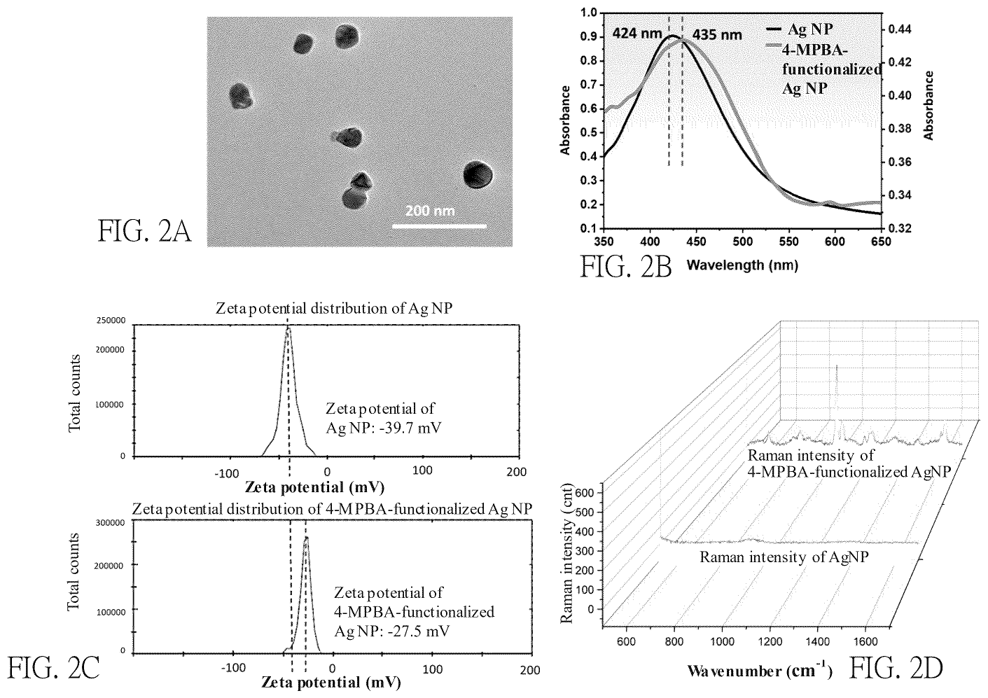

The 4-MPBA-functionalized Ag NP to be subjected to observation of morphology by transmission electron microscopy (TEM) were prepared using procedures described by Chen and Wen in an article entitled “In situ wet-cell TEM observation of gold nanoparticle motion in an aqueous solution” published in Nanoscale Res. Lett., 2012, 7:598. In brief, the 4-MPBA-functionalized Ag NP were sonicated for 10 seconds to 15 seconds, and then 10 μL of a solution containing the 4-MPBA-functionalized Ag NP were drop casted onto holey carbon films mounted on copper grids. Thereafter, the 4-MPBA-functionalized Ag NP were subjected to imaging and photography using a transmission electron microscope (Manufacturer: JEOL, Ltd., Japan; Model no.: JEM-F200) at a magnification of 30000 times. The result is shown in FIG. 2A.

FIG. 2A is a TEM image showing the surface morphology of the 4-MPBA-functionalized Ag NP. As shown in FIG. 2A, the 4-MPBA-functionalized Ag NP have uniform size distribution, and an particle size ranging from 40 nm to 60 with an average particle size thereof being 50 nm.

### 2. Light Absorbance Measurement

The Ag NP (before conjugation to the 4-MPBA) and the 4-MPBA-functionalized Ag NP were subjected to by ultraviolet-visible (UV-Vis) spectroscopy, i.e., light absorbance measurement, using a UV-Visible spectrophotometer (Manufacturer: Jasco Corp., Japan: Model no.: V-750 with integration time of 300 seconds. The result is shown in FIG. 2B.

FIG. 2B shows the UV-Vis spectra of the Ag NP and the 4-MPBA-functionalized Ag NP. As shown in FIG. 2B, the Ag NP had an absorbance peak at a wavelength of 424 nm, while the 4-MPBA-functionalized Ag NP had an absorbance peak at an increased wavelength of 435 nm, indicating a red shift of 11 nm. In addition, the decrease in absorbance intensity observed in 4-MPBA-functionalized Ag NP was due to a change in surface plasmon resonance of the Ag NP after conjugation to the 4-MPBA via thiol bonding.

### 3. Distribution of Zeta Potential

The Ag NP (before conjugation to the 4-MPBA) and the 4-MPBA-functionalized Ag NP were subjected to determination of distribution of zeta potential by dynamic light scattering using Zetasizer Nano ZS (Manufacturer: Malvern Panalytical, UK). The result is shown in FIG. 2C.

FIG. 2C shows distributions of zeta potential of the Ag NP and the 4-MPBA-functionalized Ag NP. As shown in FIG. 2C, the Ag NP had a zeta potential of −39.7 mV, and the 4-MPBA-functionalized Ag NP had an increased zeta potential of −27.5 mV. Such increase was caused by the shift in surface charge when the citrate groups of the Ag NP was replaced with the hydroxyl groups upon conjugation of the Ag NP to the 4-MPBA.

### 4. Raman Intensity

The Ag NP (before conjugation to the 4-MPBA) and the 4-MPBA-functionalized Ag NP were subjected to determination of Raman intensity by confocal Raman spectroscopy using LabRAM HR Evolution System (Manufacturer: HORIBA Scientific, Japan), which includes a Raman spectrometer mounted to a confocal microscope equipped with objective (10×). For the confocal Raman spectroscopy, the Ag NP and the 4-MPBA-functionalized Ag NP were illuminated with a laser beam having a wavelength of 532 nm and a spot diameter of 1.6 μm. The result is shown in FIG. 2D.

FIG. 2D is the Raman spectra showing the Raman intensities of the Ag NP and the 4-MPBA-functionalized Ag NP under a wavenumber ranging from 600 cm⁻¹ to 1600 cm⁻¹. As shown in FIG. 2D, a Raman peak at a wavenumber of 1078 cm⁻¹ was clearly visible for the 4-MPBA-functionalized Ag NP, whereas such peak was absent for Ag NP, indicating that the 4-MPBA-functionalized Ag NP are SERS-sensitive.

### 5. Cytotoxicity Test

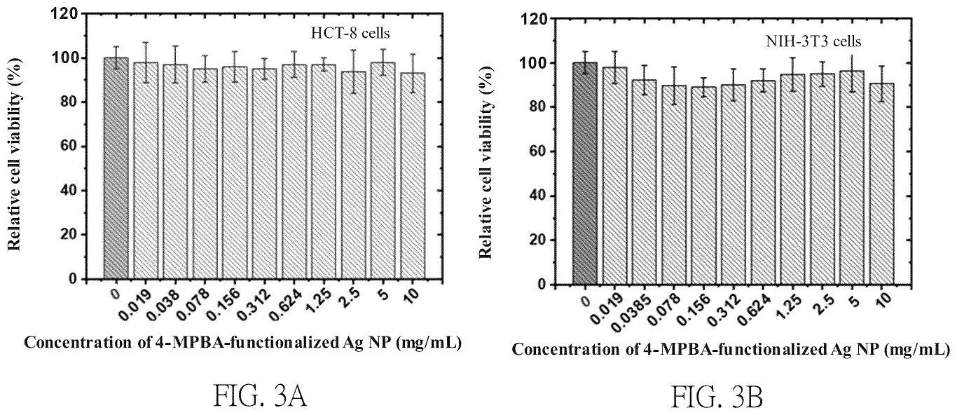

First, HCT-8 cells and NIH-3T3 cells as described in the section entitled “2. Source and cultivation of cells” of the General Experimental Materials were separately seeded at 3000 cells per well into respective wells of a 24-well culture plate containing the respective culture media, and were cultured in an incubator at 37° C. and 5% CO₂ for 48 hours. Next, the HCT-8 cells and the NIH-3T3 cells were tested against 4-MPBA-functionalized Ag NP at concentrations of 0.019 mg/L, 0.038 mg/mL, 0.078 mg/mL, 0.156 mg/mL, 0.312 mg/mL, 0.624 mg/mL, 1.25 mg/mL, 2.5 mg/mL, 5.0 mg/ml and 10.0 mg/mL, respectively, followed by cultivation at 37° C. and 5% CO₂ for 24 hours. The HCT-8 cells and NIH-3T3 cells which were not treated with 4-MPBA-functionalized Ag NP served as control. Thereafter, the culture media containing the 4-MPBA-functionalized Ag NP were removed from the respective wells, and then the HCT-8 cells and the NIH-3T3 cells were washed with phosphate-buffered saline (PBS) 3 times. After that, the cells were subjected to a cytotoxicity test utilizing a colorimetric assay using the Cell Counting Kit-8 (CCK-8) (Manufacturer: Dojindo Laboratories Co., Ltd., Japan) for determining the number of viable cells in each well. In brief, the cells in each well were incubated with 100 μL of the CCK-8 reagent for 30 minutes, and then subjected to absorbance measurement at a wavelength of 450 nm using the GloMax® Explorer Multimode Microplate Reader (Manufacturer: Promega), followed by determination of relative cell viability carried out in accordance to the manufacturer's protocol. The result is shown in FIG. 3A and FIG. 3B.

FIG. 3A and FIG. 3B are graphs showing the relative cell viability (%) of HCT-8 cells and NIH-3T3 cells, respectively. As shown in FIG. 3A and FIG. 3B, the average values for the relative cell viability for HCT-8 cells and NIH-3T3 cells, when tested against 4-MPBA-functionalized Ag NP at a concentration ranging from 0.019 mg/L to 10.0 mg/mL, were 90% and 89.78%, respectively, indicating that the 4-MPBA-functionalized Ag NP are highly biocompatible.

### 6. Cellular Uptake

Since the 4-MPBA itself is not a fluorescent molecule, a fluorescence-based Ag NP were required to be first prepared and then incubated with the cells, followed by determining whether cellular uptake of such fluorescence-based Ag NP occurs after incubation with the cells, so that the distribution and occurrence of cellular uptake of the 4-MPBA-functionalized Ag NP of the present disclosure, after being incubated with the cells, can be deduced based on the results obtained using the fluorescence-based Ag NP. Previous studies, for example, Nayak P. S. et al., in an article entitled “Lactoferrin adsorption onto silver nanoparticles interface: Implications of corona on protein conformation, nanoparticle cytotoxicity and the formulation adjuvanticity” published in J. Chem. Eng., 2019, 470-484 have reported the use of fluorescein isothiocyanate (FITC) for tracking the presence of Ag NP in the cells.

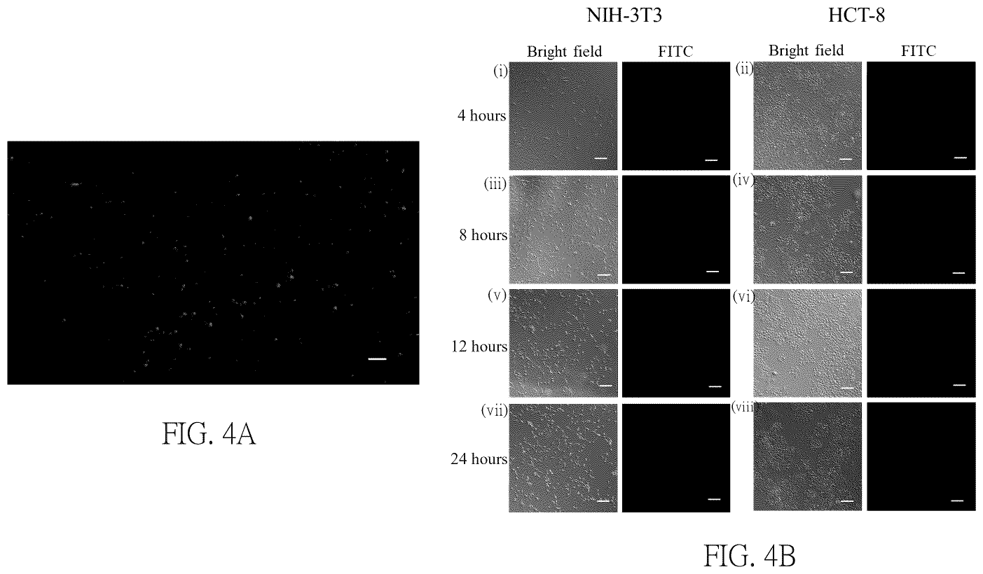

In brief, 1 mg of the Ag NP described in section A of this example were mixed with 10 μL (concentration: 1 mg/ml) of FITC dye according to the procedures described in the article by Nayak P. S. et al. as mentioned in the foregoing. Thereafter, the resultant FITC-conjugated Ag NP were subjected to imaging using a fluorescence-based confocal microscope (Manufacturer: Carl Zeiss; Model no.: LSM780) with an excitation wavelength of 495 nm and at a magnification of 20 times. The result is shown in FIG. 4A.

FIG. 4A is a fluorescence microscopy image of the FITC-conjugated Ag NP with a scale bar of 10 μm.

In order to determine whether the FITC-conjugated Ag NP will be uptaken into the cells after incubation, a time-dependent study was performed by incubating a respective one of the HCT-8 cells and the NIH-3T3 cells with the FITC-conjugated Ag NP at 37° C. and 5% CO₂. At the time points of after 4 hours, 8 hours, 12 hours and 24 hours after incubation, the HCT-8 cells and NIH-3T3 cells were respectively subjected to washing with PBS and fixing with paraformaldehyde (4%), followed by imaging by fluorescence-based confocal microscopy as described above. The result is shown in FIG. 4B.

FIG. 4B shows the bright-field and dark-field confocal fluorescence microscopy images of the HCT-8 cells and the NIH-3T3 cells after incubation with the FITC-conjugated Ag NP in which (i) and (ii) represent 4 hours of incubation, (iii) and (iv) represent 8 hours of incubation, (v) and (vi) represent 12 hours of incubation, and (vii) and (viii) represent 24 hours of incubation. As shown in FIG. 4B, no FITC fluorescence signal was detected in the dark-field images of the HCT-8 cells and the NIH-3T3 cells even after 24 hours of incubation [see right panel of (vii) and (viii)], indicating the absence of cellular uptake of the FITC-conjugated Ag NP in the HCT-8 cells and the NIH-3T3 cells.

### C. Evaluation of 4-Mercaptophenylboronic Acid (4-MPBA)-Functionalized Ag NP in Detection of Glucose in Solutions

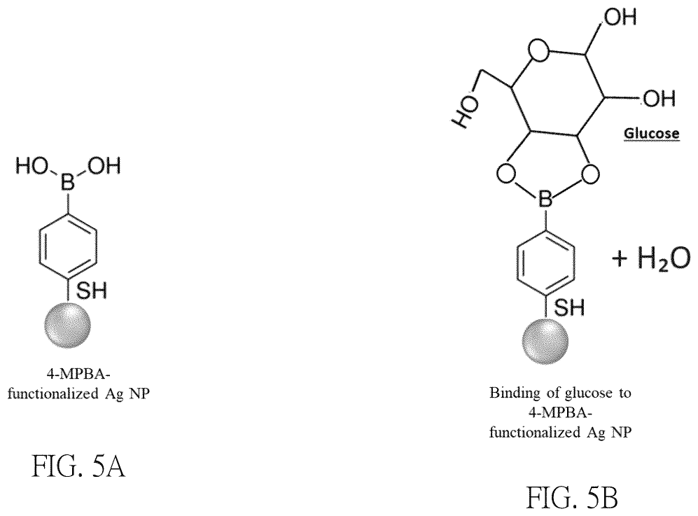

FIG. 5A is a diagram showing the chemical structure of the 4-MPBA-functionalized Ag NP, in which the 4-MPBA binds to the Ag NP via thiol bonding, while FIG. 5B is another diagram showing the chemical structure of the 4-MPBA-functionalized Ag NP with the hydroxyl (—OH) groups of the 4-MPBA binding to a glucose molecule through a dehydration reaction. The applicants proposed that, for the 4-MPBA-functionalized Ag NP of the present disclosure, binding of glucose to the OH groups of the 4-MPBA would lead to a change in the Raman intensity at the wavenumber of 1078 cm⁻¹, and that the increase in the Raman intensity is directly proportional to the concentration of glucose. In the following experiment, the 4-MPBA-functionalized Ag NP obtained in section A above were used for detecting glucose in solutions at a range of concentrations, including those of the blood glucose level.

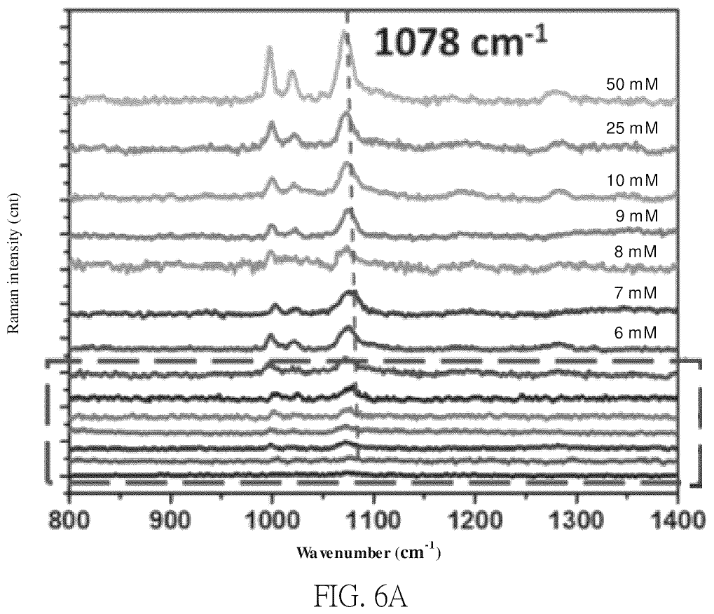

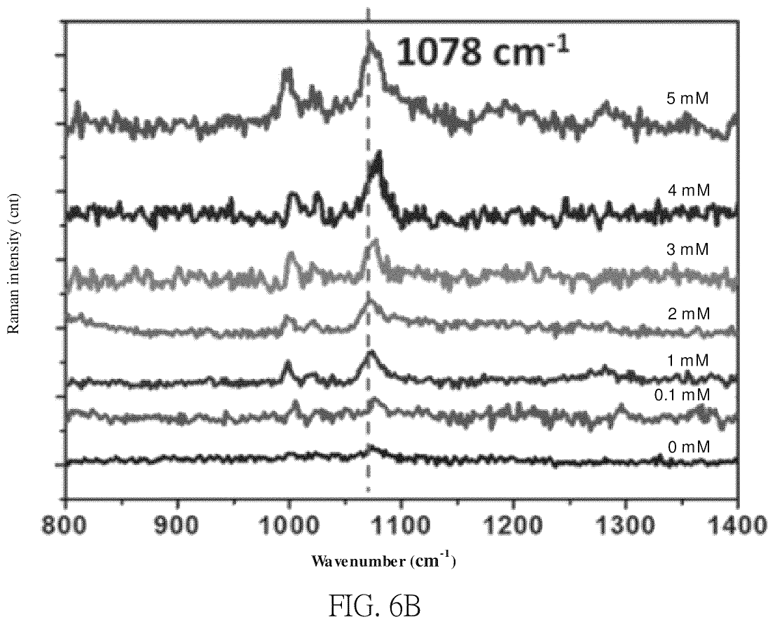

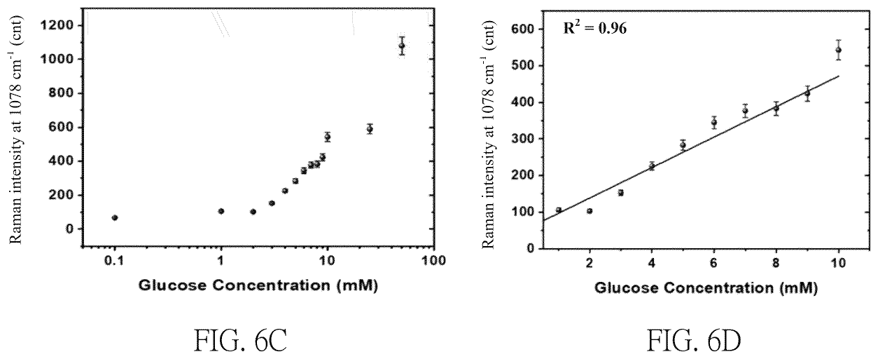

In brief, glass slides (Manufacturer: Marienfeld) were cleaned with a piranha solution (i.e., a mixture formed by mixing sulfuric acid (H₂SO₄) and hydrogen peroxide (H₂O₂) in a ratio of 3:1), and then subjected to surface modification by dipping into 5% (v/v) of 3-aminopropyltriethoxysilane (APTES) in methanol for 4 hours, followed by air-drying for 5 minutes. Next, 10 UL of the 4-MPBA-functionalized Ag NP (10 mg/mL) was drop-casted on each of the APTES-functionalized glass slide and then dried at 30° C. for 40 minutes to allow the 4-MPBA-functionalized Ag NP to be fixed on thereon. Meanwhile, a PBS (1×) solution (i.e., glucose concentration: 0 mM and serving as control) and several glucose solutions having glucose concentrations of 0.1 mM, 1 mM, 2 mM, 3 mM, 4 mM, 5 mM, 6 mM, 7 mM, 8 mM, 9 mM, 10 mM, 25 mM and 50 mM, respectively, were prepared by dissolving glucose in the PBS (1×) solution. Thereafter, 10 UL of the glucose solutions were dropped on the 4-MPBA-functionalized Ag NP on the APTES-functionalized glass slides, respectively, followed by confocal Raman spectroscopy using the LabRAM HR Evolution System (Manufacturer: HORIBA Scientific, Japan) as described in Item 4, section B above. For the confocal Raman spectroscopy, the 4-MPBA-functionalized Ag NP on the APTES-functionalized glass slides were illuminated with a laser beam having a wavelength of 532 nm and a spot diameter of 1.6 μm. The results are shown in FIG. 6A and FIG. 6B.

FIG. 6A is a Raman spectra showing the Raman intensities for the various glucose solutions having concentrations ranging from 0.1 mM to 50 mM (only 6 mM, 7 mM, 8 mM, 9 mM, 10 mM, 25 mM and 50 mM were indicated) at a wavenumber ranging from 800 cm⁻¹ to 1400 cm⁻¹ after being tested against the 4-MPBA-functionalized Ag NP. FIG. 6B is an enlarged view taken from the boxed region indicated by the dashed line in FIG. 6A which shows Raman intensities for the various glucose solutions having concentrations ranging from 0.1 mM to 5.0 mM at a wavenumber ranging from 800 cm⁻¹ to 1400 cm⁻¹ after being tested against the 4-MPBA-functionalized Ag NP. As shown in FIG. 6A and FIG. 6B, the Raman intensity increased proportionally with the increased concentrations of glucose and was indicated by the dynamic shifting of the Raman peaks at the wavenumber of 1078 cm⁻¹. The 4-MPBA-functionalized Ag NP had a relatively large emission intensity, and the hybridized Raman peaks resulted from C—S stretching coupled with C-ring breathing and bending modes of the 4-MPBA upon binding of glucose to the 4-MPBA.

Subsequently, the data shown in FIG. 6A and FIG. 6B were extracted and interpreted as linear graphs, in which the Raman intensities at the wavenumber of 1078 cm⁻¹ were indicated in the Y-axis while the glucose concentrations were indicated in the X-axis. Curve fitting of these data were conducted using the Origin Pro 8.5 software (OriginLab Corporation).

FIG. 6C is a graph showing the Raman intensities at the wavenumber of 1078 cm⁻¹ plotted against a glucose concentration ranging from 0.1 mM to 50 mM. FIG. 6D is another graph showing the Raman intensities at the wavenumber of 1078 cm⁻¹ plotted against a glucose concentration ranging from 0.1 mM to 10.0 mM with a linear regression, R² of 0.96. The plot shown in FIG. 6D would be used as a calibration curve for determining glucose concentrations corresponding to the Raman intensities at the wavenumber of 1078 cm⁻¹, which will be described hereinafter.

The limit of detection (LoD) of the glucose concentration was calculated in accordance to the International Union of Pure and Applied Chemisty (IUPAC) guidelines using the following formula (1):

$$L o D = {B}_{0} + 3 \times standard deviation of {B}_{0} \qquad ( 1 )$$

wherein B₀=Raman intensity of the blank signal (without glucose)

The value of B₀ was calculated to be 54.4±4, while the standard deviation of B₀ was 4. Thus, the Raman intensity of the LoD was 66.4, and based on the plot shown in FIG. 6D, the LoD for the glucose concentration was determined to be 0.1 mM. These result indicate that the 4-MPBA-functionalized Ag NP of the present disclosure exhibit a linear response within the physiological range of glucose concentration.

### D. Evaluation of 4-Mercaptophenylboronic Acid (4-MPBA)-Functionalized Ag NP in Terms of Repeated Detection of Glucose in Solutions

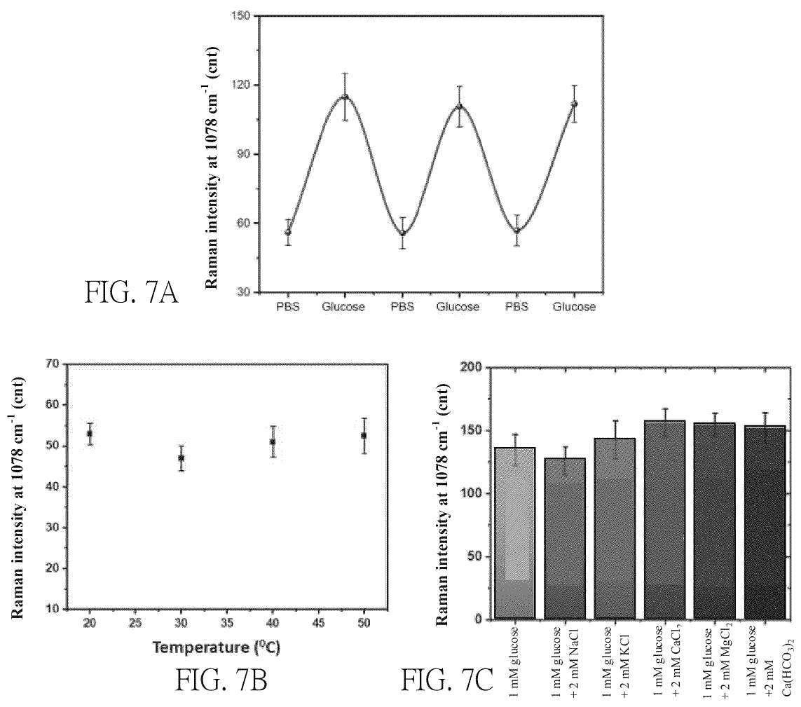

In order to determine the reproducibility of the aforesaid result with respect to the detection of glucose in solutions by the 4-MPBA-functionalized Ag NP of the present disclosure, the 4-MPBA-functionalized Ag NP on the APTES-functionalized glass slides, a PBS solution, (i.e., glucose concentration: 0 mM) and a glucose solution (glucose concentration: 1 mM) were prepared according to the procedures described in section C above. Next, the PBS solution and the glucose solution were sequentially tested against the 4-MPBA-functionalized Ag NP on the APTES-functionalized glass slides for 3 times (each test was 30 seconds) with the time interval between the PBS solution and the glucose solution tested being 50 seconds to 60 seconds, followed by confocal Raman spectroscopy as described in section C of this example above so as to determine the Raman intensity at the wavenumber of 1078 cm⁻¹. The result is shown in FIG. 7A.

FIG. 7A is a Raman spectrum showing the Raman intensity at a wavenumber of 1078 cm⁻¹ for the PBS solution and the glucose solution after such solutions are sequentially tested 3 times against the 4-MPBA-functionalized Ag NP. As shown in FIG. 7A, the Raman intensity changed dynamically with the glucose solution having a relatively high Raman intensity and the PBS solution having a relatively low Raman intensity, and the values of these Raman intensities remained consistent despite these two solutions being alternated 3 times. This result indicates that the binding of glucose to the 4-MPBA-functionalized Ag NP of the present disclosure is reversible.

### E. Evaluation of 4-Mercaptophenylboronic Acid (4-MPBA)-Functionalized Ag NP in Terms of Thermal Stability, and Specificity Against Metallic Elements and Non-Metallic Compounds in Detection of Glucose in Solutions

In order to determine the thermal stability of by the 4-MPBA-functionalized Ag NP of the present disclosure, the 4-MPBA-functionalized Ag NP on the APTES-functionalized glass slides were prepared according to the procedures described in section C of this example. Next, the 4-MPBA-functionalized Ag NP on the APTES-functionalized glass slides were placed on a hotplate having a temperature preset at 20° C., followed immediately by confocal Raman spectroscopy as described in section C of this example above so as to determine the Raman intensity at the wavenumber of 1078 cm⁻¹. These procedures were repeated with the temperatures of the hotplate being preset at 30° C., 40° C. and 50° C., respectively. The result is shown in FIG. 7B.

FIG. 7B is a graph showing the Raman intensity at the wavenumber of 1078 cm⁻¹ for the 4-MPBA-functionalized Ag NP at a temperature ranging from 20° C. to 50° C. As shown in FIG. 7B, at the temperatures of 20° C., 30° C., 40° C. and 50° C., there was no significant difference in the Raman intensity at the wave number of 1078 cm⁻¹ for the 4-MPBA-functionalized Ag NP, and the relative standard deviation from the mean Raman intensity was merely 2.73%, indicating that the 4-MPBA-functionalized Ag NP of the present disclosure exhibited thermal stability in such range of temperature.

Determination of interference by metal ions might be helpful to understand the impact of the 4-MPBA-functionalized Ag NP of the present disclosure on cellular processes. It should be noted that metal ions, such as sodium (Na⁺) and potassium (K⁺), have been reported to influence glucose regulation by exerting their effects in various physiological processes. Thus, in order to determine whether different types of metallic and non-metallic compounds interferes with the function of the 4-MPBA-functionalized Ag NP of the present disclosure, several glucose solutions with or without metal ions were prepared, i.e., a first glucose solution containing 1 mM of glucose, a second glucose solution containing 1 mM of glucose and 2 mM NaCl, a third glucose solution containing 1 mM of glucose and 2 mM of KCl, a fourth glucose solution containing 1 mM of glucose and 2 mM of CaCl₂), a fifth glucose solution containing 1 mM of glucose and 2 mM of MgCl₂, and a sixth glucose solution containing 1 mM of glucose and 2 mM of Ca(HCO₃)₂. Meanwhile, the 4-MPBA-functionalized Ag NP on the APTES-functionalized glass slides were prepared according to the procedures described in section C of this example. Next, the first to the sixth glucose solutions were respectively tested against the 4-MPBA-functionalized Ag NP on the APTES-functionalized glass slides, followed by confocal Raman spectroscopy as described in section C of this example. The result is shown in FIG. 7C.

FIG. 7C is a graph showing the Raman intensity at the wave number of 1078 cm⁻¹ for the first to the sixth glucose solutions respectively containing 1 mM of glucose, 1 mM of glucose and 2 mM NaCl, 1 mM of glucose and 2 mM of KCl, 1 mM of glucose and 2 mM of CaCl₂), 1 mM of glucose and 2 mM of MgCl₂, and 1 mM of glucose and 2 mM of Ca(HCO₃)₂, after being tested against the 4-MPBA-functionalized Ag NP on the APTES-functionalized glass slides. As shown in FIG. 7C, there was no significant difference in the Raman intensity at the wavenumber of 1078 cm⁻¹ for the aforesaid glucose solutions when tested against the 4-MPBA-functionalized Ag NP, demonstrating that the presence of different types of metallic and non-metallic compounds in the glucose-containing solutions does not affect the function of the 4-MPBA-functionalized Ag NP of the present disclosure. This result indicate that the 4-MPBA-functionalized Ag NP of the present disclosure are highly reliable.

### Example 2. Preparation and Characterization of 3D Tumor Spheroid with 4-MPBA-Functionalized Ag NP Embedded Therein

Since results obtained in Example 1 demonstrate that the 4-MPBA-functionalized Ag NP of the present disclosure are SERS-sensitive, highly biocompatible, thermally stable, highly reliable, have reproducibility with respect to the detection of glucose in vitro, and demonstrate a linear response within the physiological range of glucose concentration, such 4-MPBA-functionalized Ag NP are deemed suitable to be incorporated into a 3D culture model. In this example, a 3D culture model, in the form of a 3D tumor spheroid, was established by first preparing a microwell array chip, and then using the microwell array chip to cultivate a mixture of the 4-MPBA-functionalized Ag NP and a co-culture of colon cancer cells and fibroblasts so as to obtain 3D tumor spheroids with the 4-MPBA-functionalized Ag NP embedded therein, followed by characterization of such 3D tumor spheroids.

### A. Preparation of PDMS-Based Microwell Array Chip

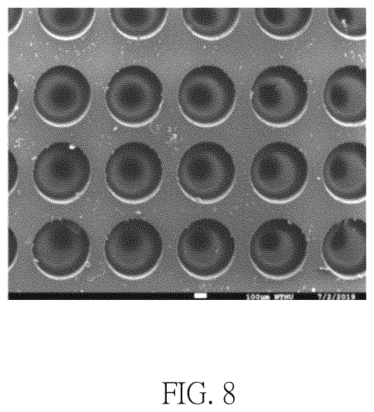

The microwell array chip was prepared by a soft lithography process in accordance to the procedures described by Goudar et al., in an article entitled “Impact of a desmoplastic tumor microenvironment for colon cancer drug sensitivity: a study with 3D chimeric tumor spheroids” published in ACS Appl. Mater. Interfaces, 2021, Vol. 13, p. 48478-48491. In brief, SU-8 2150 was coated on a 4-inches silicon wafer using a spin coater at a speed of 1000 rpm for 30 seconds. Next, a mask including a pattern of microwells was aligned onto the soft-baked wafer, followed by UV exposure to transfer the pattern of microwells. Thereafter, the wafer was developed using SU-8 photoresist for 25 minutes, thereby forming a silicon mold containing microwells (SU-8 pillars). Meanwhile, silicon elastomer and curing agent were mixed in a volume ratio of 10:1 to form a polydimethylsiloxane (PDMS) mixture, followed by a degassing the PDMS mixture to prevent formation of microbubbles. Then, the PDMS mixture was poured on the silicon mold containing SU-8 pillars, followed by a curing process conducted at 65° C. for 2 hours. After that, a PDMS-based microwell array chip was peeled off from the silicon mold, followed by treating the PDMS-based microwell array chip with 1 wt % of Pluronic F-127 (i.e., a copolymer surfactant dissolved in distilled water) for 24 hours. Use of the Pluronic F-127 might minimize cell adhesion and facilitate tumor spheroid formation. The PDMS-based microwell array chip, with a total number of microwells being 600, was subjected to scanning electron microscopy (SEM) using an ultra-high resolution Schottky Field Emission Scanning Electron Microscope (Manufacturer: JEOL, Ltd., Japan; Model no.: JSM-7610F) at a magnification of 40 times. The results are shown in FIG. 8.

FIG. 8 is an SEM image illustrating a top schematic view of several microwells of the PDMS-based microwell array chip, with a scale bar of 100 μm. As shown in FIG. 8, the microwells had a circular shape, a diameter of 500 μm with a gap of 100 μm between two adjacent ones of the microwells, and a well depth of approximately 500 μm.

### B. Preparation and Characterization of 3D Tumor Spheroid Formed Using the PDMS-Based Microwell Array Chip

The PDMS-based microwell array chip obtained in section A of this example was used for cultivating a mixture containing 4-MPBA-functionalized Ag NP, the human ileocecal adenocarcinoma HCT-8 cells, and mouse embryonic fibroblast NIH-3T3 cells. In brief, 100 μL of 4-MPBA-functionalized Ag NP (concentration: 10 mg/mL) prepared as described in section A of Example 1 were added to a co-culture of the HCT-8 cells and NIH-3T3 cells which were described in the section “2. Source and cultivation of cells” of the General Experimental Materials and which were mixed at a ratio of cell number of 1:1, followed by seeding at approximately 45 to 50 cells into respective microwells (containing RPMI 1640 and DMEM culture media in a volume ratio of 1:1) of the PDMS-based microwell array chip, followed by incubation in an incubator at 37° C. and 5% CO₂ for 5 days, so as to allow formation of the 3D tumor spheroid with the 4-MPBA-functionalized Ag NP embedded therein. The 3D tumor spheroid formed from the co-culture of HCT-8 cells and the NIH-3T3 cells was subjected to the following evaluation described hereinafter.

### 1. Growth of 3D Tumor Spheroid

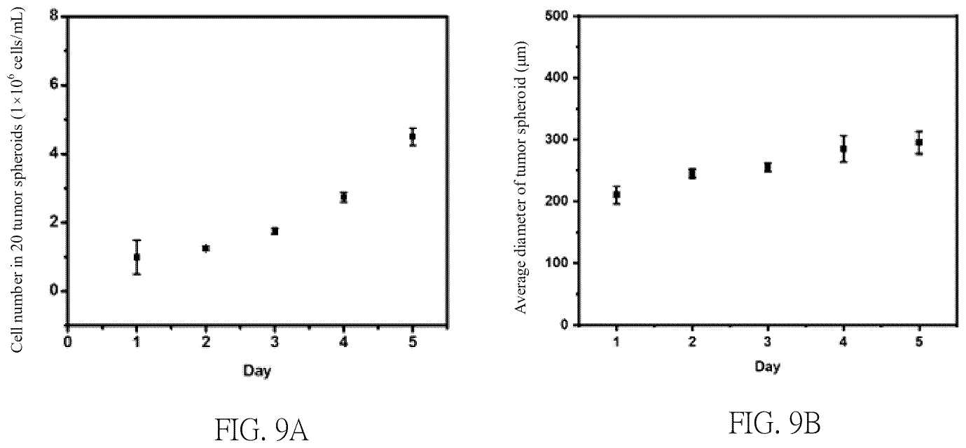

The 3D tumor spheroids grown in the PDMS-based microwell array chip were subjected to determination of growth for a time period of 5 days. In brief, on each day of the 5-day time period, i.e., on day 1, day 2, day 3, day 4 and day 5 of incubation after seeding of the cells, bright field images of the 3D tumor spheroids in the microwells of the PDMS-based microwell array chip were captured using an inverted microscope (Olympus IX70) equipped with a CCD camera (Manufacturer: Micro-shot Technology Co., Ltd., China) at a magnification of 4 times, followed by analyzing the bright field images using Image J software (Developer: National Institutes of Health, USA) so as to determine the size of the 3D tumor spheroids during the period of growth. After that, twenty 3D tumor spheroids were pipetted out from the microwells without disrupting the 3D structure thereof, and then placed into a microcentrifuge tube (Manufacturer: Eppendorf). Next, the culture media was removed, and the 3D tumor spheroids were washed with PBS. Then, 50 μL of 0.25% trypsin-disodium ethylenediaminetetraacetic acid (EDTA) was added to the 3D tumor spheroids, followed by incubation at 37° C. for 5 to 7 minutes. After adding 1 mL of culture media, the cells were gently resuspended using a pipette to completely disrupt the 3D structure, so as to separate the cells. After that, the cells were counted using LUNA-II™ Automated Cell Counter. The results are shown in FIG. 9A and FIG. 9B.

FIG. 9A is a graph showing the cell number per twenty 3D tumor spheroids determined on day 0 to day 5. FIG. 9B is a graph showing the average size of the 3D tumor spheroid (per twenty tumor spheroids) determined on day 1 to day 5 with the corresponding bright field images of one of the 3D tumor spheroids inserted therein. As shown in FIG. 9A, the cell number increased exponentially from day 1 to day 5, and as shown in FIG. 9B, the average size of the 3D tumor spheroids increased steadily from day 1 to day 5. These results indicate that the 3D tumor spheroid exhibited an exponential growth.

### 2. Distribution of HCT-8 Cells and NIH-3T3 Cells in 3D Tumor Spheroid

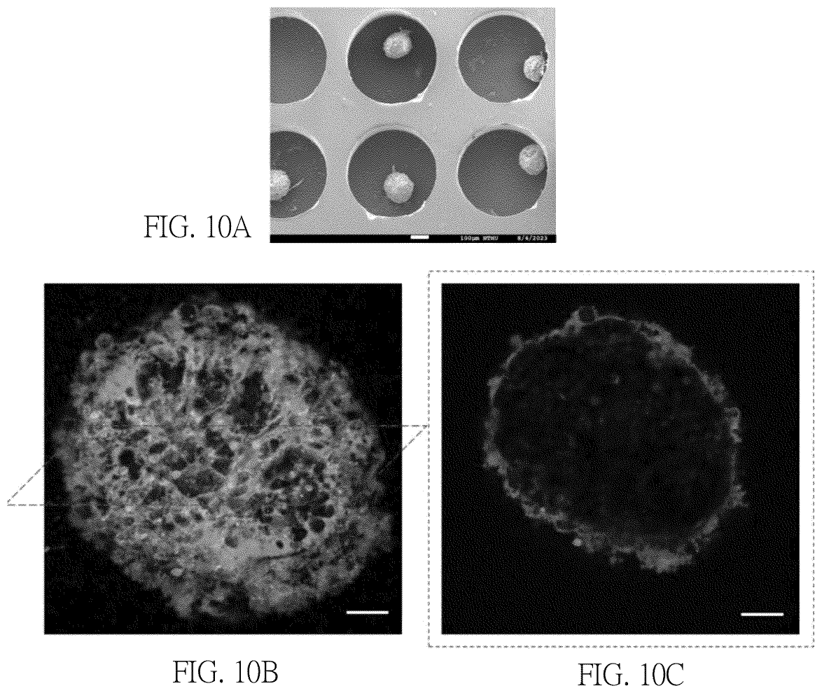

After 48 hours of incubation of the HCT-8 cells, NIH-3T3 cells and 4-MPBA-functionalized Ag NP in the microwells of the PDMS-based microwell array chip, the 3D tumor spheroids formed therein were also subjected to scanning electron microscopy performed using field emission scanning electron microscope (Manufacturer: JEOL, Ltd., Japan; Model no.: JSM-6500 F). The result is shown in FIG. 10A.

FIG. 10A is an SEM image showing the 3D tumor spheroids formed in the microwells of the PDMS-based microwell array chip after incubating the HCT-8 cells, NIH-3T3 cells and 4-MPBA-functionalized Ag NP therein for 48 hours. As shown in FIG. 10A, one 3D tumor spheroid was formed in each microwell of the PDMS-based microwell array chip, indicating that use of Pluronic F-127 for coating the PDMS-based microwell array chip minimizes cell adhesion and facilitates formation of 3D tumor spheroid.

In order to determine the distribution of the HCT-8 cells and NIH-3T3 cells in the 3D tumor spheroid, HCT-8 cells and NIH-3T3 cells were subjected to fluorescent labeling using CellTracker™ Red CMTPX dye and CellTracker™ Green CMFDA dye, respectively, so as to obtain HCT-8 pre-labeled cells and NIH-3T3 pre-labeled cells. These pre-labeled cells were subjected to growth so as to form 5-day old 3D tumor spheroids, followed by imaging using a confocal laser scanning microscope (Manufacturer: Carl Zeiss; Model no.: LSM780) to obtain the fluorescence image of the 5-day old 3D tumor spheroids using the maximum intensity projection. The results are shown in FIG. 10B and FIG. 10C.

FIG. 10B is a fluorescence microscopy image of the 5-day old 3D tumor spheroid obtained using the maximum intensity projection, and FIG. 10C is another fluorescence microscopy image showing a cross-sectional view of the 5-day old 3D tumor spheroid which was taken from the boxed region indicated by the dotted lines in FIG. 10B. As shown in FIG. 10B and FIG. 10C, a majority of the NIH-3T3 cells (represented by the green fluorescence) were distributed at the peripheral region of the 3D tumor spheroid, whereas most of the HCT-8 cells were distributed at the core region of the 3D tumor spheroid, with a small fraction of the NIH-3T3 cells and the HCT-8 cells being distributed throughout the tumor spheroid.

### 3. Presence of 4-MPBA-Functionalized Ag NP in 3D Tumor Spheroid

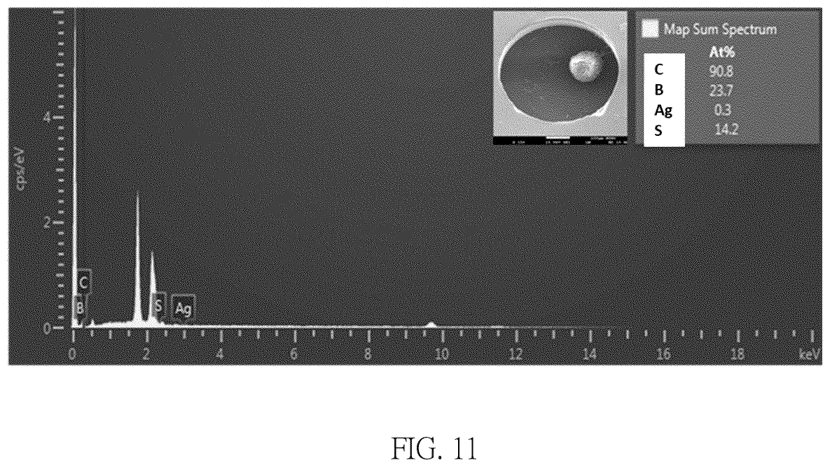

In order to determine the presence of the 4-MPBA-functionalized Ag NP in the 3D tumor spheroid, five of the 3D tumor spheroids were subjected to elemental analysis by energy-dispersive X-ray spectroscopy (EDS) analysis using an ultra-high resolution Schottky Field Emission Scanning Electron Microscope (Manufacturer: JEOL, Ltd., Japan; Model no.: JSM-7610F) at a magnification of 150 times. The result is shown in FIG. 11.

FIG. 11 is an EDS spectrum showing the elements present in the 3D tumor spheroids. As shown in FIG. 11, silver (Ag), sulfur(S), boron (B) and carbon (C) elements were present in the 3D tumor spheroid, thus confirming the presence of the 4-MPBA-functionalized Ag NP in the 3D tumor spheroid.

### 4. Distribution and Cellular Uptake of 4-MPBA-Functionalized Ag NP in 3D Tumor Spheroid

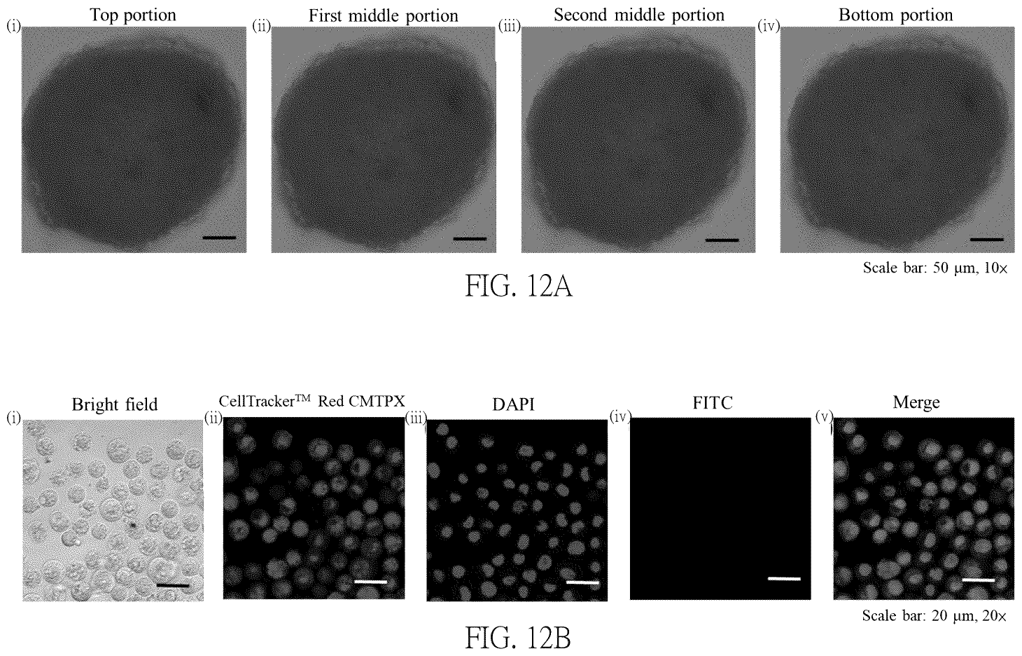

Since the 4-MPBA is not a fluorescent molecule, the distribution of the 4-MPBA-functionalized Ag NP in the 3D tumor spheroid is deduced based on the distribution of FITC-conjugated Ag NP in the 3D tumor spheroid. In brief, 100 μL of the FITC-conjugated Ag NP (concentration: 10 mg/mL) prepared according to the procedures described in the item, entitled “6. Cellular uptake” of Section B of Example 1 were added to a co-culture of HCT-8 cells and NIH-3T3 cells which were mixed at a ratio of cell number of 1:1, followed by seeding at approximately 45 to 50 cells into the respective microwells (containing RPMI 1640 and DMEM culture media in a volume ratio of 1:1) of the PDMS-based microwell array chip, followed by incubation in an incubator at 37° C. and 5% CO₂ for 5 days, so as to allow formation of the 3D tumor spheroid with the FITC-conjugated Ag NP embedded therein. Thereafter, the 3D tumor spheroids were washed using PBS (1×), fixed using paraformaldehyde (4%), and then subjected to fluorescence-based confocal microscopy using a confocal laser scanning microscope (Manufacturer: Carl Zeiss; Model no.: LSM780) with an excitation wavelength of 495 nm and at a magnification of 20 times. The results are shown in FIG. 12A.

FIG. 12A shows confocal fluorescence microscopy images of a 3D tumor spheroid formed after incubation of the HCT-8 cells, NIH-3T3 cells and FITC-conjugated Ag NP for 5 days, in which (i) to (iv) respectively are the top portion, the first middle portion, the second middle portion and the bottom portion of the 3D tumor spheroid. As shown in FIG. 12A, the FITC-conjugated Ag NP were present at different portions of the 3D tumor spheroid, indicating that the FITC-conjugated Ag NP were evenly distributed throughout the 3D tumor spheroid. These results suggest that the 4-MPBA-functionalized Ag NP of the present disclosure embedded in the 3D tumor spheroid were evenly distributed throughout the 3D tumor spheroid.

As described in the foregoing, whether cellular uptake of the 4-MPBA-functionalized Ag NP occurs in the 3D tumor spheroid was deduced based on the results obtained from the 3D tumor spheroid with the FITC-conjugated Ag NP embedded therein. In brief, five of the 5-day old 3D tumor spheroids were taken out from the microwells of the PDMS-based microwell array chip and placed into a microcentrifuge tube (Manufacturer: Eppendorf). Next, 100 μL of 0.25% trypsin-EDTA was added to 5-day old 3D tumor spheroids with the FITC-conjugated Ag NP embedded therein, followed by incubation at 37° C. for 25 minutes. Next, resuspension was conducted several times by rigorous pipetting to disrupt the 3D structure of the tumor spheroid, and then the resultant HCT-8 cells and NIH-3T3 cells were washed 3 times with PBS. Thereafter, the cell nuclei were stained with DAPI for 15 minutes, and the cell membranes of the cells were stained with CellTracker™ Red CMPTX for 30 minutes. Then, the cells were subjected to the aforesaid fluorescence-based confocal microscopy at a magnification of 20 times. These results are shown in FIG. 12B.

FIG. 12B shows are confocal fluorescence microscopy image of the HCT-8 cells and NIH-3T3 cells after disruption of the 3D structure of the 5-day old tumor spheroids, in which (i) is a bright-field image of the HCT-8 cells and NIH-3T3 cells, (ii) to (iv) are dark-field images of the HCT-8 cells and NIH-3T3 cells respectively stained with CellTracker™ Red CMPTX, DAPI and FITC dyes, and (v) is an image obtained by merging the images shown in (ii) to (iv). As shown in FIG. 12B, only the CellTracker™ Red CMPTX fluorescence signal and DAPI fluorescence signal were observed in the dark-field images of the HCT-8 cells and the NIH-3T3 cells [see (ii) and (iii)], whereas no FITC fluorescence signal can be detected in the dark-field image [see (iv)] and even after the dark-field images shown in (ii) to (iv) were merged [see (v)], indicating the absence of cellular uptake of the 4-MPBA-functionalized Ag NP in the 3D tumor spheroids. These results suggest that the 4-MPBA-functionalized Ag NP of the present disclosure embedded in the 3D tumor spheroid were present in the extracellular region of the same.

### Example 3. Determination of Glucose Concentration in 3D Tumor Spheroid and Intracellular Region of the Same, and in 3D Fibroblast Spheroid

The 4-MPBA-functionalized Ag NP of the present disclosure have been shown to be embedded in and evenly distributed throughout the 3D tumor spheroid that is formed from a co-culture of cancer cells and fibroblasts. Thus, in this example, 3D tumor spheroids with the 4-MPBA-functionalized Ag NP embedded therein were prepared and then subjected to confocal Raman spectroscopy to evaluate whether the 4-MPBA-functionalized Ag NP incorporated in the 3D tumor spheroid could be utilized as a SERS-based glucose nanosensor for determining glucose concentration in the 3D tumor spheroid (i.e., intratumor parts). In addition, the glucose concentration in the intracellular region of the 3D tumor spheroid was also determined.

For comparison purposes, 3D fibroblast spheroids were also prepared, and then subjected to confocal Raman spectroscopy so as to determine the glucose concentration in such spheroid.

### A. Determination of Glucose Concentration in 3D Tumor Spheroid and 3D Fibroblast Spheroid

The 3D tumor spheroids were prepared according to the procedures as described in Section B of Example 2. The procedures for preparing the 3D fibroblast spheroids were substantially similar to those of the 3D tumor spheroids, except that only the 4-MPBA-functionalized Ag NP and the NIH-3T3 cells were seeded at approximately 80 to 100 cells in each microwell of the microwell array chip.

In brief, on a respective one of day 3 and day 5 after seeding the 4-MPBA-functionalized Ag NP and the respective cells into the microwells of the microwell array chip, a respective one of the five 3D tumor spheroids and five 3D fibroblast spheroids (out of a total of approximately 600 spheroids) thus formed were pipetted out from the microwells of the microwell array plate to be transferred onto a well-like region of a pre-clean glass slide (size: 26 mm×76 mm). The well-like region with a height of was approximately 250 μm was formed by stacking two hollow ring-shaped tapes (Manufacturer: Wen Lung Printing Industry Co., Ltd., Taiwan) having a thickness of approximately 250 μm on the glass slide. Next, 50 μL of a mounting gel (Manufacturer: ScyTek Laboratories, Taiwan) was added to a respective one of the 3D tumor spheroids and the 3D fibroblast spheroids, and then these spheroids were covered using a cover slip (size: 18 mm×18 mm) thereon, followed by sealing the glass slide with the respective spheroids mounted thereon using a transparent nail polish.

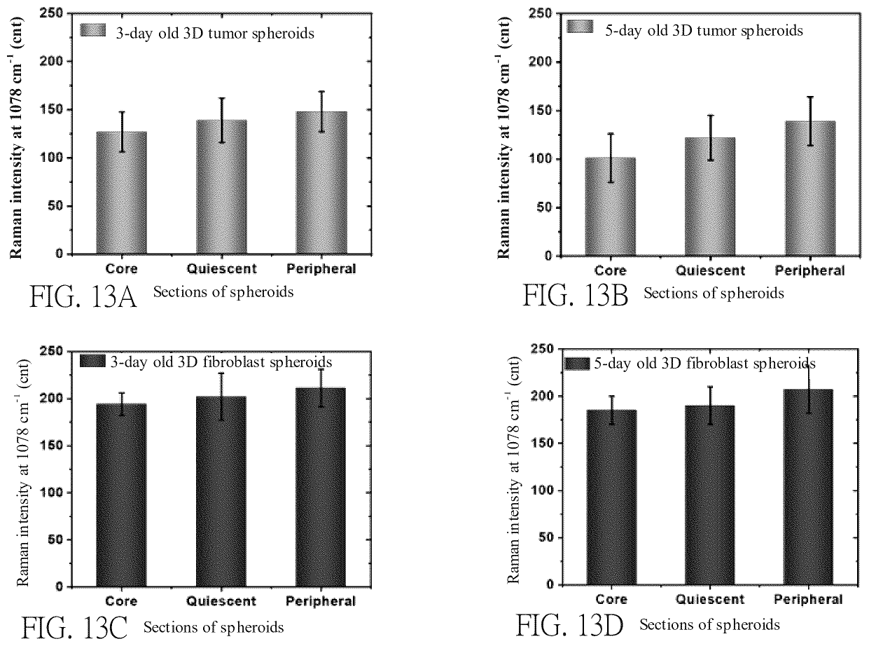

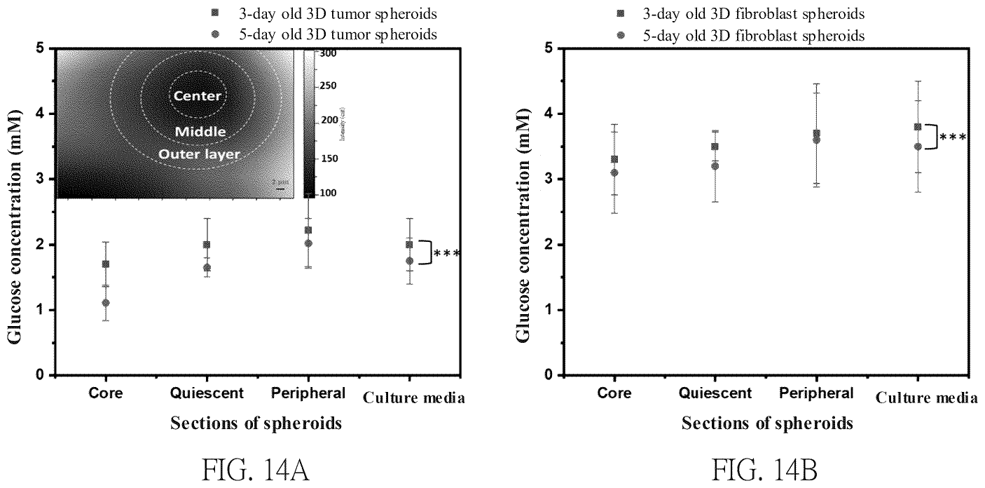

Thereafter, a respective one of the 3D tumor spheroids and the 3D fibroblast spheroids were subjected to confocal Raman spectroscopy using the LabRAM HR Evolution System (Manufacturer: HORIBA Scientific, Japan) as described in Item 4, section B of Example 1. For the confocal Raman spectroscopy, the 3D tumor spheroids and the 3D fibroblast spheroids were illuminated with a laser beam having a wavelength of 532 nm and a spot diameter of 1.6 μm. The Raman spectrometer has diffraction gratings with resolutions equal to 600 lines/mm. For the confocal Raman spectroscopy, a respective one of the 3D tumor spheroids and the 3D fibroblast spheroids were subjected full 3D sectional scanning performed at a room temperature of 22° C. on three sections of the spheroids, i.e., core, quiescent and peripheral sections, from the top to bottom portions of each spheroid with a step size of 10 μm interval across the z-axis with calculation being conducted on an average of 15 slices and 50 intensity points in each sections (approximately 100 μm), and the Raman mapping images of the spheroid indicating distribution of Raman intensities thereof at a wavenumber bandwidth of 1078 cm⁻¹ were generated by point-to-point mapping using LabSpec 6 spectroscopy suite software (Developer: HORIBA Scientific, Japan) based on a two-dimensional matrix array fixed upon the bright field image of the spheroid. Each Raman mapping image was divided into 3 portions, i.e., center, middle and outer layer, which respectively correspond to the core, quiescent and core sections of the spheroids. Afterwards, the Raman intensities (unit: counts) for each sections of the spheroids were extracted from the Raman mapping image using the color-coded bar attached thereto, and plotted as graphs. The results are shown in FIG. 13A to FIG. 13D.

FIG. 13A and FIG. 13B are graphs showing the Raman intensities extracted and determined from the Raman mapping images that were generated at the wavenumber bandwidth of 1078 cm⁻¹ for the core, quiescent and peripheral sections of the 3-day old 3D tumor spheroids and the 5-day 3D tumor spheroids, respectively. FIG. 13C and FIG. 13D are graphs showing the Raman intensities extracted and determined from the Raman mapping images that were generated at the wavenumber bandwidth of 1078 cm⁻¹ for the core, quiescent and peripheral sections of the 3-day old 3D fibroblast spheroids and the 5-day old 3D fibroblast spheroids, respectively.

As shown in FIG. 13A and FIG. 13B, for the 3-day old and 5-day old 3D tumor spheroids, the Raman intensities determined in the peripheral sections were the highest and those in the core sections were the lowest, suggesting formation of a glucose concentration gradient between the peripheral sections and the core sections of the 3D tumor spheroids. As shown in FIG. 13C and FIG. 13D, for the 3-day old and 5-day old 3D fibroblast spheroids, the Raman intensities determined in the peripheral sections were the highest and those in the core sections were the lowest, suggesting formation of a glucose concentration gradient between the peripheral sections and the core sections of the 3D fibroblast spheroids. These results will be discussed in detail hereinafter with the results shown in FIG. 14A and FIG. 14B.

The thus extracted Raman intensities determined in the core, quiescent and peripheral sections of 3-day old and 5-day old 3D tumor spheroids and those of the 3-day old and 5-day old 3D fibroblast spheroids were mapped to the calibration curve as described in section C of Example 1 and shown in FIG. 6D, so as to determine the corresponding glucose concentrations of the same. The results are shown in FIG. 14A and FIG. 14B.

FIG. 14A is a graph showing the glucose concentrations determined in the core, quiescent and peripheral sections of the 3-day old 3D tumor spheroids and those of the 5-day old 3D tumor spheroids, and the Raman mapping image of one of the 5-day old 3D tumor spheroids (scale bar: 2 μm) indicating the distribution of Raman intensity at the wavenumber of 1078 cm⁻¹ was shown in the insert of this figure. As shown in FIG. 14A, for the 3-day old 3D tumor spheroids, the glucose concentrations in the peripheral section and the core section were 2.22 mM and 1.7 mM, respectively, while for the 5-day old 3D tumor spheroids, the glucose concentrations in the peripheral section and the core section were 2.02 mM and 1.1 mM, respectively, demonstrating that a glucose concentration gradient formed between the peripheral sections and the core sections was greater for the 5-day old 3D tumor spheroids (i.e., 0.95 mM) compared with that for the 3-day old 3D tumor spheroids (i.e., 0.52 mM). Formation of the glucose concentration gradient may be attributed to the desmoplastic tumor microenvironment in the 3-day old and 5-day old 3D tumor spheroids. It should be noted that, for both the 3-day old and 5-day old tumor spheroids, the glucose concentration in the peripheral section was higher compared to that in the quiescent and core section because the cells at the peripheral section of the tumor spheroid were proximal to the culture media surrounding the 3D tumor spheroid and thus, were able to obtain a greater amount of nutrient from the culture media compared to the cells at the quiescent and core sections of the tumor spheroid, and possibly because the 4-MPBA-functionalized Ag NP, which were utilized as the SERS-based glucose sensor in this experiment, were present in the extracellular region of the 3D tumor spheroid, while the cells at the core section of the tumor spheroid faced shortages of nutrient.

In addition, the glucose concentrations determined in the core, quiescent and peripheral sections of the 5-day old 3D tumor spheroids and in the culture media thereof decreased significantly compared to those of the 3-day old 3D tumor spheroids and in the culture media thereof, indicating an increased cellular uptake of glucose by the tumor cells (i.e., increased glucose consumption) with the increase in the number of days of growth so as to support the growth of the 3D tumor spheroids. In other words, the glucose concentration present in the intracellular region of the 5-day old 3D tumor spheroids would be expected to be higher than that of the 3-day old 3D tumor spheroids (see also the result shown in Table 1 of section B of this example to be described hereinafter).

FIG. 14B is a graph showing the glucose concentrations determined in the core, quiescent and peripheral sections of the 3-day old 3D fibroblast spheroids and those of the 5-day old 3D fibroblast spheroids. As shown in FIG. 14B, for the 3-day old 3D fibroblast spheroids, the glucose concentrations in the peripheral section and the core section were 3.7 mM and 3.3 mM, respectively, while for the 5-day old 3D fibroblast spheroids, the glucose concentrations in the peripheral section and the core section were 3.6 mM and 3.1 mM, respectively, demonstrating that a glucose concentration gradient formed between the peripheral sections and the core sections was slightly greater for the 5-day old 3D fibroblast spheroids (i.e., 0.5 mM) compared with that for the 3-day old 3D fibroblast spheroids (i.e., 0.4 mM). In addition, the glucose concentration determined in the culture media for the 5 day-old 3D fibroblast spheroids decreased significantly compared to that in the culture media for the 3 day-old 3D fibroblast spheroids, indicating that there was an increase in cellular uptake of glucose by the fibroblasts (i.e., increased glucose consumption) with the increase in the number of days of growth in order support the growth of the 3D fibroblast spheroids.

Taken together, the results in FIG. 14A and FIG. 14B demonstrate that the glucose concentration gradients measured from the core sections to the peripheral sections of the 3-day old and 5-day old 3D tumor spheroids were more prominent compared with those of the 3-day old and 5-day old 3D fibroblast spheroids, and that the glucose concentrations determined in each of the core, quiescent and peripheral sections (i.e., intratumor sections) of the 3D tumor spheroids were lower compared to those of the 3D fibroblast spheroids because the glucose consumption by a combination of the HCT-8 cells and the NIH-3T3 cells was higher compared to that of the NIH-3T3 cells alone.

### B. Determination of Glucose Concentration in Intracellular Region of 3D Tumor Spheroid

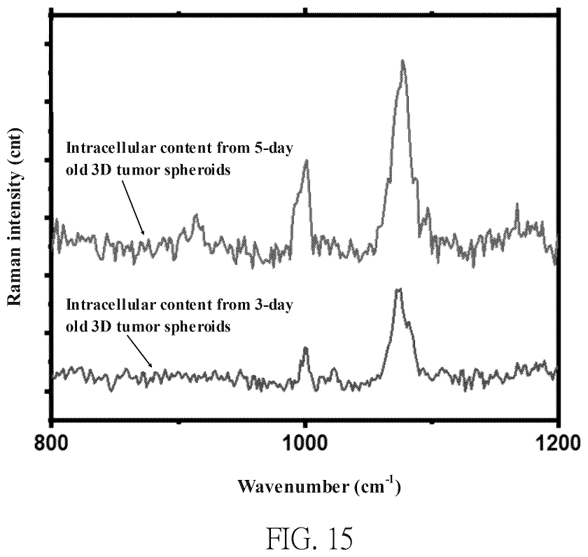

In order to gain insight on glucose consumption in the 3D tumor spheroids, the following experiment was conducted to measure glucose concentration in the intracellular region of the 3D tumor spheroids. In brief, a respective one of twenty 3-day old 3D tumor spheroids and twenty 5-day old 3D tumor spheroids were pipetted out from the microwells of the microwell array chip without disrupting the 3D structure thereof, and then placed into a microcentrifuge tube (Manufacturer: Eppendorf). Next, the culture media was removed, and the 3D tumor spheroids were washed with PBS. Then, 50 μL of 0.25% trypsin-EDTA was added to the 3D tumor spheroids, followed by incubation at 37° C. for 5 minutes to 7 minutes. Thereafter, the cells were resuspended using a pipette to completely disrupt the 3D structure, and then Triton X-100 (10% v/v) was added to permeabilize the cell wall so as to extract intracellular content. Then, centrifugation was performed at a speed of 1200 rpm for 5 minutes at 4° C., and the resultant supernatant (i.e., liquid portion) containing the intracellular content of the 3D tumor spheroids was collected. Meanwhile, 4-MPBA-functionalized Ag NP on the APTES-functionalized glass slides were prepared as according to the procedures described in section C of Example 1. Subsequently, the thus collected supernatants containing the intracellular contents of the 3-day old 3D tumor spheroids and the 5-day old 3D tumor spheroids, respectively, were tested against the 4-MPBA-functionalized Ag NP on the APTES-functionalized glass slides, and then subjected to confocal Raman spectroscopy according to the procedures described in section C of Example 1, so as to obtain Raman spectra for the intracellular contents of these tumor spheroids. The result is shown in FIG. 15.

FIG. 15 is a Raman spectra showing the Raman intensities for the intracellular contents of the 3-day old 3D tumor spheroids and the 5-day old 3D tumor spheroids, respectively, under a wavenumber ranging from 800 cm⁻¹ to 1200 cm⁻¹. As shown in FIG. 15, the Raman intensity for the intracellular content of the 5-day old 3D tumor spheroids was higher compared to that of the 3-day old 3D tumor spheroids.

Next, thus extracted Raman intensities determined for the intracellular contents of the 3-day old 3D tumor spheroids and those of the 5-day old 3D tumor spheroids were respectively mapped to the calibration curve as described in section C of Example 1 and shown in FIG. 6D, so as to determine the corresponding glucose concentrations of the same. The results are shown in Table 1 below.

TABLE 1

| | Intracellular content of | Intracellular content of |
|---|---|---|
| | 3-day old 3D tumor | 5-day old 3D tumor |
| | spheroids | spheroids |
| Glucose | 2.13 | 4.32 |
| concentration (mM) | | |

As shown in Table 1, the glucose concentration determined in the intracellular content of the 5-day old 3D tumor spheroids was approximately 2-fold higher compared with that of the 3-day old 3D tumor spheroids, indicating that there was an increase in the amount of glucose stored in the intracellular region of the 3D tumor spheroids as the size of the 3D tumor spheroids increased due to growth. Thus, this result supports the finding of the decrease in glucose concentrations in the extracellular region of the 3-day old and 5-day old 3D tumor spheroids and in the culture media as shown in FIG. 14A above.

In summary, the aforesaid results suggest that by embedding the 4-MPBA-functionalized Ag NP to serve as a SERS-based glucose nanosensor in 3D tumor spheroids that are formed from a co-culture of human colon cancer cells and fibroblasts, the method of the present disclosure is capable of continuously and rapidly detecting glucose in such 3D tumor spheroids in vitro, and thus real time measurement of glucose concentration in different sections of such 3D tumor spheroids can be performed in a non-invasive manner. As such, the method for determining glucose concentration in 3D tumor spheroids of the present disclosure enhances the understanding of glucose distribution and/or metabolism in the tumor microenvironment in vivo.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for determining glucose concentration in a three-dimensional tumor spheroid in vitro, comprising the steps of:
(a) adding a surface-enhanced Raman spectroscopy (SERS)-based nanosensor including 4-mercaptophenylboronic acid (4-MPBA)-functionalized silver nanoparticles to a co-culture of cancer cells and stromal cells so as to form a first mixture;
(b) adding the first mixture into microwells of a polydimethylsiloxane (PDMS)-based microwell array chip;
(c) subjecting the first mixture in the PDMS-based microwell array chip to incubation at 37° C. so as to form the three-dimensional tumor spheroid with the SERS-based glucose nanosensor embedded therein;
(d) transferring the three-dimensional tumor spheroid onto a glass slide to allow the three-dimensional tumor spheroid to be mounted thereon;
(e) subjecting the three-dimensional tumor spheroid mounted on the glass slide to confocal Raman spectroscopy so as to obtain Raman mapping images of the three-dimensional tumor spheroid; and
(f) extracting Raman intensities from the Raman mapping images and mapping the Raman intensities to a calibration curve so as to determine glucose concentration in the three-dimensional tumor spheroid in vitro.

2. The method as claimed in claim 1, wherein in step (a), the cancer cells are selected from the group consisting of colon cancer cells, breast cancer cells, lung cancer cells, ovarian cancer cells, pancreatic cancer cells, prostate cancer cells, skin cancer cells, bone marrow cancer cells, brain cancer cells, gastrointestinal cancer cells, renal cancer cells, liver cancer cells, testicular cancer cells, bladder cancer cells, cervical cancer cells, esophageal cancer cells, endometrial cancer cells, and combinations thereof, and the stromal cells are selected from the group consisting of endothelial cells, macrophages, fibroblasts, mesenchymal stem cells, osteoblasts, chondrocytes, myocytes, and combinations thereof.

3. The method as claimed in claim 2, wherein the colon cancer cells are human ileocecal adenocarcinoma HCT-8 cells, and the fibroblasts are mouse embryonic fibroblast NIH-3T3 cells.

4. The method as claimed in claim 3, wherein a ratio of a number of the human ileocecal adenocarcinoma HCT-8 cells to a number of the mouse embryonic fibroblast NIH-3T3 cells is 1:1.

5. The method as claimed in claim 1, wherein in step (a), a concentration of the SERS-based nanosensor added to the co-culture ranges from 0.019 mg/mL to 10.0 mg/mL.

6. The method as claimed in claim 5, wherein in step (a), the concentration of the SERS-based nanosensor added to the co-culture is 10.0 mg/mL.

7. The method as claimed in claim 1, wherein in step (c), the first mixture in the PDMS-based microwell array chip is incubated at 37° C. for a time period ranging from 48 hours to 120 hours.

8. The method as claimed in claim 7, wherein in step (c), the first mixture in the PDMS-based microwell array chip is incubated at 37° C. for 72 hours.

9. The method as claimed in claim 7, wherein step (c), the first mixture in the PDMS-based microwell array chip is incubated at 37° C. for 120 hours.

10. The method as claimed in claim 1, wherein in step (a), the 4-MPBA-functionalized silver nanoparticles are prepared by the steps of:
(i) heating a silver nitrate solution at 90° C. under stirring to obtain a boiled silver nitrate solution;
(ii) adding dropwise a sodium citrate solution to the boiled silver nitrate solution under stirring so as to form a yellow-colored solution;
(iii) cooling the yellow-colored solution to room temperature until a colloidal solution containing silver nanoparticles was formed;
(iv) adding a 4-MPBA solution in ethanol, which is prepared by mixing 4-MPBA and ethanol, to the colloidal solution containing the silver nanoparticles under stirring so as to form a second mixture; and
(v) incubating the second mixture in the dark at 4° C. to allow the 4-MPBA to be conjugated to the silver nanoparticles, so as to obtain the 4-MPBA-functionalized silver nanoparticles.

11. The method as claimed in claim 10, wherein step in (ii), the sodium citrate solution is added dropwise to the boiled silver nitrate solution for a time period ranging from 1 minute to 15 minutes.

12. The method as claimed in claim 11, wherein in step (ii), the sodium citrate solution is added dropwise to the boiled silver nitrate solution for 15 minutes.

13. The method as claimed in claim 10, wherein in step (iv), a weight ratio of the silver nanoparticles to the 4-MPBA ranges from 533:1 to 650:1.

14. The method as claimed in claim 13, wherein in step (iv), the weight ratio of the silver nanoparticles to the 4-MPBA is 650:1.

15. The method as claimed in claim 10, wherein in step (v), the second mixture is incubated in the dark for a time period ranging from 8 hours to 12 hours.

16. The method as claimed in claim 15, wherein in step (v), the second mixture is incubated in the dark for 12 hours.

17. The method as claimed in claim 10, wherein the 4-MPBA-functionalized silver nanoparticles have a particle size ranging from 40 nm to 60 nm.

18. The method as claimed in claim 17, wherein the 4-MPBA-functionalized silver nanoparticles have an average particle size of 50 nm.
